(19) United States
(12) Patent Application Publication
(10) Pub. No.: US 2018/0256073 A1
(43) Pub. Date: Sep. 13, 2018

(54) Gait Analysis Through Computerized Sole Inspection

(71) Applicant: Enda Athletic Inc., Dover, DE (US)

(72) Inventors: Weldon Kennedy, Englewood, NJ (US); Charles Hudak, New York, NY (US); Brian Bjelovuk, New York, NY (US)

(21) Appl. No.: 15/457,032

(22) Filed: Mar. 13, 2017

(51) Int. Cl. A61B 5/11; G06T 7/00; G06K 9/48

(52) U.S. Cl. CPC A61B 5/112; A43D 1/08; G06K 9/48; G06T 7/0002

(57) ABSTRACT

Disclosed herein are system, method, and computer program product embodiments for analyzing a person's gait through image analysis of the person's footwear. An embodiment operates by receiving an image associated with a shoe sole, the image displaying one or more cells forming part of a tread pattern of the shoe sole. The system then determining, based on the image, the wear levels of the cells and analyzes the wear levels to determine a wear pattern of the shoe sole. The system transmits information associated with the wear pattern for display in an interface of a computing device.

### BACKGROUND

### Technical Field

Embodiments generally relate to computerized image analysis and the design of shoe soles.

### Background

Human gait analysis relates to the systematic study of human locomotion through observation—augmented by instrumentation—for measuring body movements, body mechanics, and the activity of the muscles. For example, gait analysis may be used to assess, plan, and treat individuals with conditions affecting their ability to walk. Gait analysis is also commonly used in sports biomechanics to help athletes perform more efficiently and to identify posture-related or movement-related problems.

Certain running shoe designers have recently adopted rudimentary gait analysis techniques in marketing and selling their products. For example, some running stores offer camera-based or weight-sensor based systems to analyze customer's feet anatomy or gait pattern to try to recommend suitable footwear. However, these techniques are often not accurate and do not result in meaningful guidance for runners.

### SUMMARY

Disclosed herein are system, method, and computer program product embodiments for analyzing a person's gait through image analysis of the person's footwear. An embodiment operates by receiving an image associated with a shoe sole, the image displaying one or more cells forming part of a tread pattern of the shoe sole. The system then determining, based on the image, the wear levels of the cells and analyzes the wear levels to determine a wear pattern of the shoe sole. The system transmits information associated with the wear pattern for display in an interface of a computing device.

The embodiments disclosed above are only examples, and the scope of this disclosure is not limited to them. Particular embodiments may include all, some, or none of the components, elements, features, functions, operations, or steps of the embodiments disclosed above. Embodiments according to the invention are in particular disclosed in the attached claims directed to a method, a storage medium, a system and a computer program product, wherein any feature mentioned in one claim category, e.g. method, can be claimed in another claim category, e.g. system, as well. The dependencies or references back in the attached claims are chosen for formal reasons only. However any subject matter resulting from a deliberate reference back to any previous claims (in particular multiple dependencies) can be claimed as well, so that any combination of claims and the features thereof are disclosed and can be claimed regardless of the dependencies chosen in the attached claims. The subject-matter which can be claimed comprises not only the combinations of features as set out in the attached claims but also any other combination of features in the claims, wherein each feature mentioned in the claims can be combined with any other feature or combination of other features in the claims. Furthermore, any of the embodiments and features described or depicted herein can be claimed in a separate claim and/or in any combination with any embodiment or feature described or depicted herein or with any of the features of the attached claims.

### BRIEF DESCRIPTION OF THE DRAWINGS

The accompanying drawings are incorporated herein and form a part of the specification.

FIG. 1 illustrates a gait analysis system 100, according to an example embodiment.

FIG. 2. illustrates an example embodiment of a client device configured to transmit an image of a shoe sole to a gait analysis service.

FIG. 3 illustrates an example shoe sole tread for performing image processing gait analysis, according to an example embodiment.

FIG. 4 illustrates a zoomed-in detailed view of a portion of an array of cells forming part of an example shoe sole tread, according to an example embodiment.

FIG. 5 illustrates a cross-section of a cell with annotations highlighting its multiple levels, according to an example embodiment.

FIG. 6 shows an example method for performing gait analysis using image processing of a shoe sole, according to an example embodiment.

FIG. 7 illustrates an example computer system 700.

In the drawings, like reference numbers generally indicate identical or similar elements. Additionally, generally, the left-most digit(s) of a reference number identifies the drawing in which the reference number first appears.

### DETAILED DESCRIPTION

Provided herein are system, method and/or computer program product embodiments, and/or combinations and sub-combinations thereof, for analyzing a person's gait through image analysis of the person's footwear.

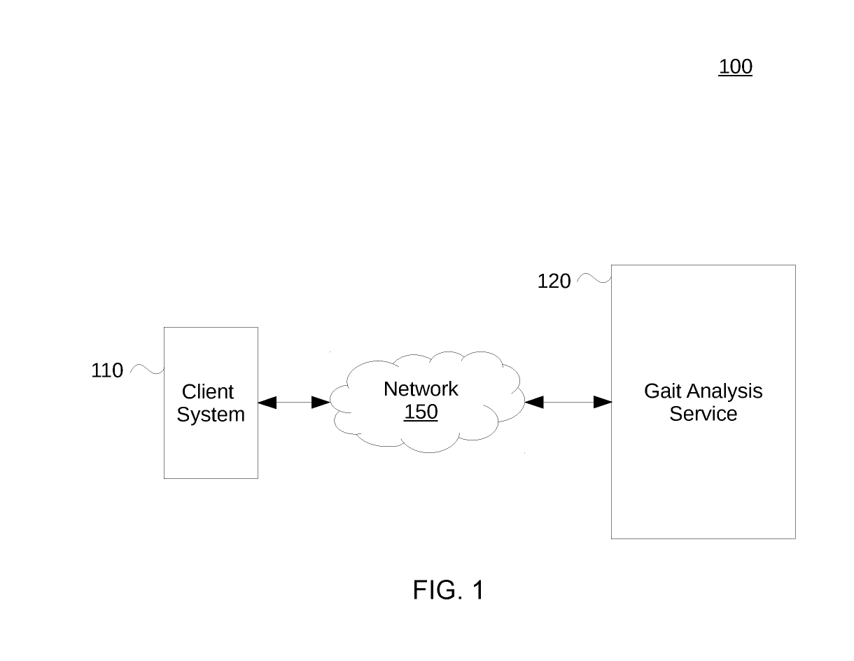

FIG. 1 illustrates a gait analysis system 100, according to an example embodiment. The system may include a client computing device 110 and a gait analysis service 120 connected through a network 150. Client computing device 110 may include any combination of software and computing devices suitable for interacting and communicating with gait analysis service 120, such as, by way of example, a personal computer, mobile computer, laptop computer, mobile phone, smartphone, personal digital assistant, tablet computer, wearable device, etc. Gait analysis service 120 may include combination of software and computing devices suitable to provide server and user interfacing functionalities, such as, by way of example, server computers, database systems, storage area networks, web servers, application servers, etc., or any combination thereof. The network 150 may be any communications network suitable for transmitting data between computing devices, such as, by way of example, a Local Area Network (LAN), a Wide Area Network (WAN), Metropolitan Area Network (MAN), Personal Area Network (PAN), the Internet, wireless networks, satellite networks, overlay networks, cellular networks, or any combination thereof.

In particular embodiments, a user of client device 110 may input a digital image of a shoe sole (of a shoe that has been used for running or walking) through a user interface provided by client device 110. The user interface may be provided in any suitable manner, such as, by way of example, an application, a webpage, a mobile application, a web application, etc. In particular embodiments, client device 110 is a smartphone, and the user may capture the image using a camera integrated into the smartphone to take a picture of the bottom of a shoe, as directed by a mobile application. In particular embodiments, a user may transfer the image through other means to client device 110.

In particular embodiments, client device 110 may transmit the digital image to gait analysis service 120. Gait analysis service 120 may then perform a digital image processing of the digital image to perform a gait analysis. As an example, gait analysis service 120 may use any suitable digital image processing algorithm to determine which portions of a sole have worn out more than others, one or more angles of wearing, etc. In an embodiment, system 100 is entirely contained within client device 110, such that it does not include a network 150, and client device 110 performs all functionalities of gait analysis service 120 described in this disclosure.

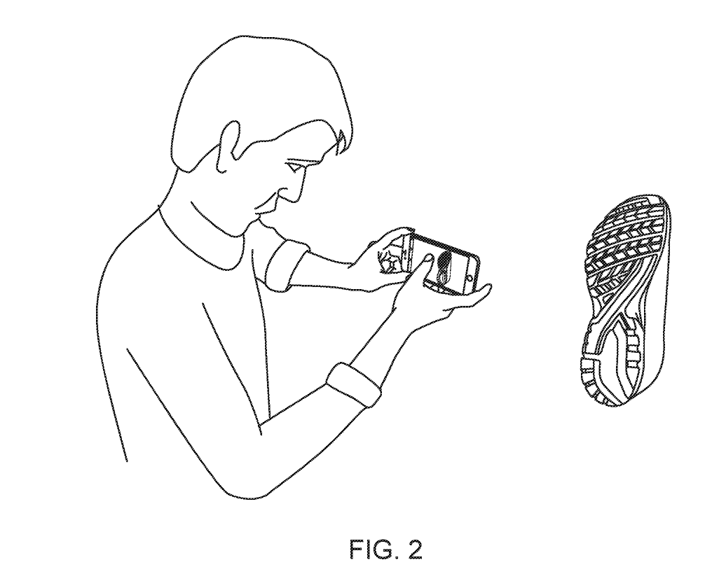

FIG. 2. illustrates an example embodiment of a client device 110 configured to transmit an image of a shoe sole to a gait analysis service 120. As shown in this example, a user may use a smartphone to take a picture of her shoe soles and transmit the image to gait analysis service 120. In an example, the smartphone may receive and display information associated with the results of the analysis.

Once the image processing is performed, gait analysis service 120 may associate the sole with a gait profile based on the detected wear patterns of the sole. As an example, gait analysis service 120 may compare an image of the runner's sole with a sample from a professional runner, to be used as an exemplar. Gait analysis service 120 may then determine areas with additional wear relative to the exemplar to detect issues in the runner's stride. As an example, gait analysis service 120 may determine 1) where a runner is striking while running, 2) the amount of over-stride 3) any wear that occurs from rotation while the foot is planted. Based on the comparison, gait analysis system 120 may determine, for example, that a sole has excessive wear in the heel and light wear on the forefoot, indicating a likelihood that the user has an over-stride and a heel strike. In another example, gait analysis system 120 may determine the sole has excessive wear on the latter forefoot, indicating a likelihood of an over-stride with a forefoot strike. In another example, gait analysis system 120 may determine a sole has excessive wear in the center of the forefoot and through to the big toe, indicating a likelihood of excessive rotation once the foot is planted. While the comparison and analysis of sole wear patterns has been described in particular manners, this disclosure contemplates comparing and analyzing sole wear patterns in any suitable manner.

In particular embodiments, a user of client device 110 may communicate via network 150 with gait analysis service 120 to create a user profile. The profile may be maintained by gait analysis service 120, which may provide a graphical user interface for interacting with the profile information. The profile and interface created through any suitable methods of maintaining online profiles, such as a website or application using a standard web registration for creating a username and password for logging in and accessing the profile.

In an embodiment, a user may submit one or more images of soles as explained above and associate them with the user profile. As such, the gait profile identified by the image processing, as explained above, may be associated with the user profile. In this manner, gait analysis service 120 may provide personalized information, marketing, recommendations, etc. that are custom-tailored to the user's running gait profile. For example, gait analysis service 120 may provide a description of the user's gait analysis and its effects of the person's body (feet, knees, back, etc.), along with recommendations on how to correct any problems (e.g., “Your running gait is likely to affect your knees. We recommend you switch to biking every other day.”) In another example, gait analysis service 120 may provide suggestions for how to run differently, what part of the foot to step, how to move arms, etc (e.g., “We recommend that you land with your heels when running.”). In an example, gait analysis service 120 may congratulate the runner and encourage them to keep running in great form. In another example, gait analysis service 120 may provide recommendations of particular shoes that may suit the user based on the gait profile, and may also provide links, offers, or other marketing materials for the user to buy the shoes. In another example, gait analysis service 120 may provide links and recommendations of particular coaches that may be able to assist a user in improving her running gait.

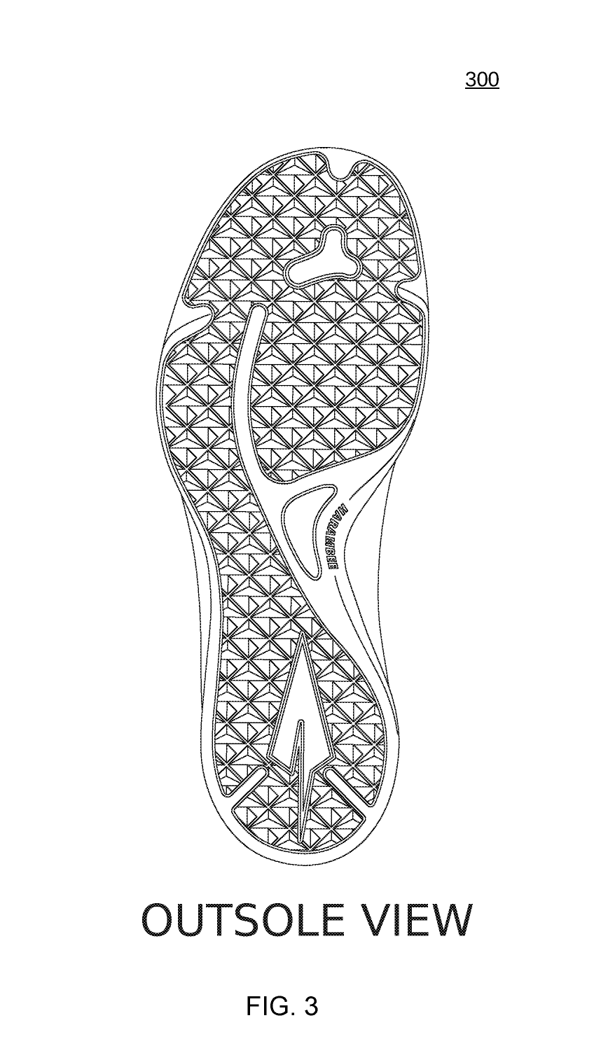

FIG. 3 illustrates an example shoe sole tread 300 for performing image processing gait analysis, according to an example embodiment. In an embodiment, the image of a shoe sole that a user of client device 110 inputs into gait analysis system 120 is an image of a shoe sole 300 specially designed for image processing. As an example, the user may use a shoe with shoe sole 300 to run for some amount of time or distance (e.g., 10 miles, by way of example). After the user has run with the shoe, the bottom of the shoe sole may have presumably worn out to some extent. The user then inputs an image of the worn-out shoe into gait analysis system 120 as explained above.

In an embodiment, shoe sole 300 is made of any material or combination of materials suitable for making shoe soles, such as, by way of example and not limitation, synthetic rubber, natural rubber, carbon rubber, solid rubber, gum rubber, Duralon, ethylene vinyl acetate (EVA), wood, leather, cork, etc.

In particular embodiments, shoe sole 300 is comprised of an array of cells 310 divided by ridges 320, as shown in the example of FIG. 3. In particular embodiments, every cell 310 is of substantially equal size and ridges 320 are of substantially equal width. In this manner, an image processing process may be able to identify with precision each cell 310 of shoe sole 300.

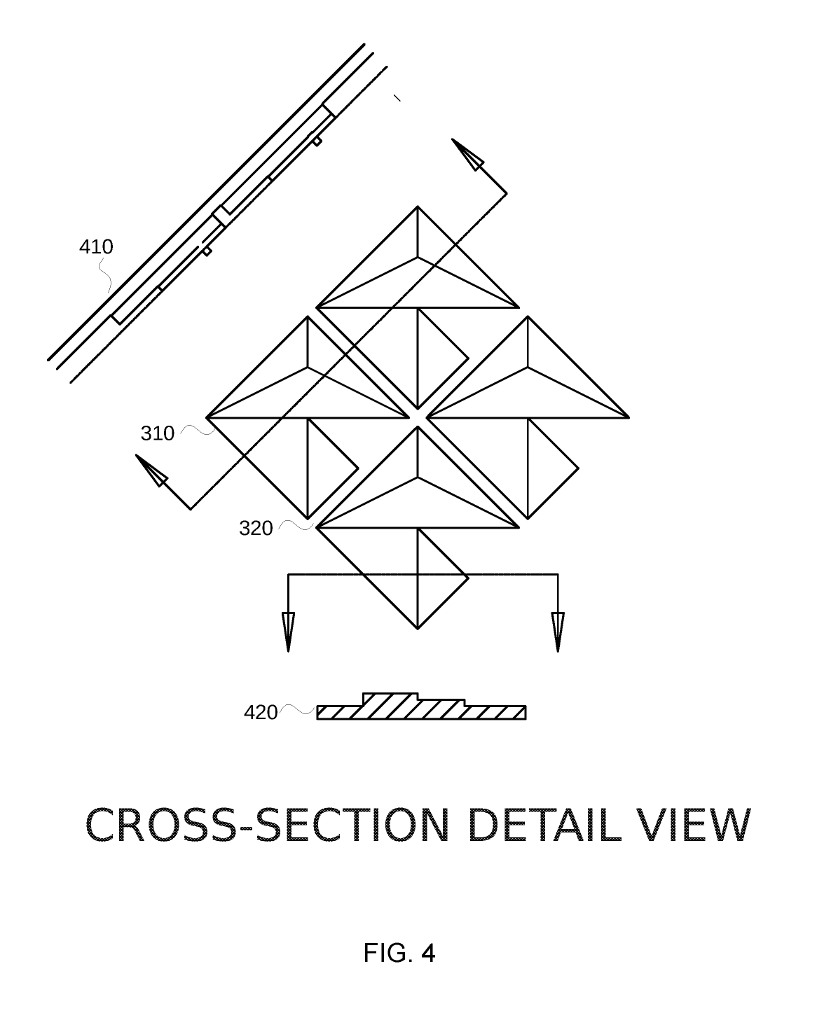

FIG. 4 illustrates a zoomed-in detailed view of a portion of the array of cells 310. FIG. 4 also shows a first cross-section 410 and a second cross-section 420 of the shoe sole in the directions illustrated by the respective arrows. In an embodiment, each cell 310 in the array may include multiple segments of different height. As shown in the example cross-sections of FIG. 4, each cell may have various levels of vertical steps, in a stair-like fashion. When a shoe cell 310 is not worn, these levels may be visually detected by the eye, or by an image processing algorithm. When a shoe cell 310 wears out, a level that is a step above vertically may wear out to the level of a neighboring level, thus making the step disappear. In this manner, an image processing system may detect the level of wear of a cell through visual inspection.

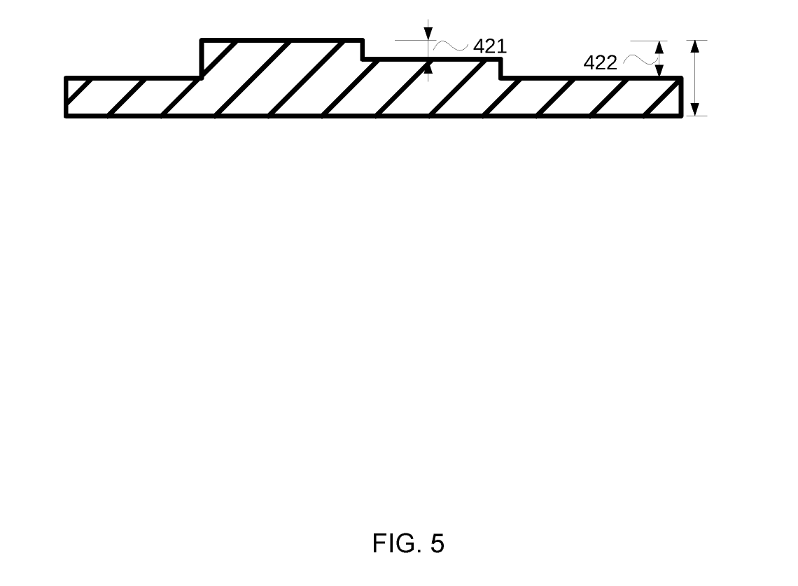

FIG. 5 illustrates the cross-section 420 of a cell 310 with annotations highlighting its multiple levels, according to an example embodiment. In an example, each cell 310 may have two or more segments (e.g., segments 311 and 312), each segment having a different height or level. In an example, cross-section 420 of cell 310 shows that the cell may comprise segments of unequal height when in a non-used state, i.e., before being used for running or walking. As the shoe gets used, a first height 421 of a top level segment may begin to decrease as the top rubber wears off. As the top level wears-off, a visual cue at the border between the first and second segments may change. When the top level wears-off completely, then the first and second segments may then be at the same level, and thus the visual border between them disappears. Thus, an image processing system of gait analysis service 120 may detect the level of wear of a cell 310 by determining the presence or absence of the visual lines dividing segments from a vertical point of view.

In particular embodiments, each segment of a cell 310 may comprise a particular shape. An image processing algorithm may have prior knowledge of the shape and height of each segment, and with this information may thus be able to determine the amount of wear of each cell.

The wearing of a shoe may be measured more precisely by using additional levels in each cell. For example, a cell that has worn off to a second height 422 would indicate a higher level of wear than one that has worn off to height 421. In an embodiment, an image processing system may assign a number or category to each cell 310 of an array of cells forming a shoe sole. As an example, each cell may be a assign a wear-level of NEW, SLIGHT WEAR, MODERATE WEAR, HEAVY WEAR, etc. In another example, each cell may be assigned a number, e.g., 1-5, describing the wear level. In an embodiment, the number of wear categories corresponds to a number of distinct physical steps in the cell, and the assign category corresponds to the number of steps that have worn off.

In particular embodiments, the shape of the cells may be used to determine a rotation of a foot while running or walking. As an example, a cell 310 may include segments that border each other at various angles, such as orthogonal or diagonal with respect to each other, as shown in FIG. 4. In particular embodiments, a border or step between two segments may wear unequally depending on the angle at which the foot strikes the ground when a person is running. For example, one border between two segments may only partially wear of (e.g., half of the border wears off). In this manner, an image processing algorithm may detect the rotation of a foot based on the particular pattern of wear of the steps.

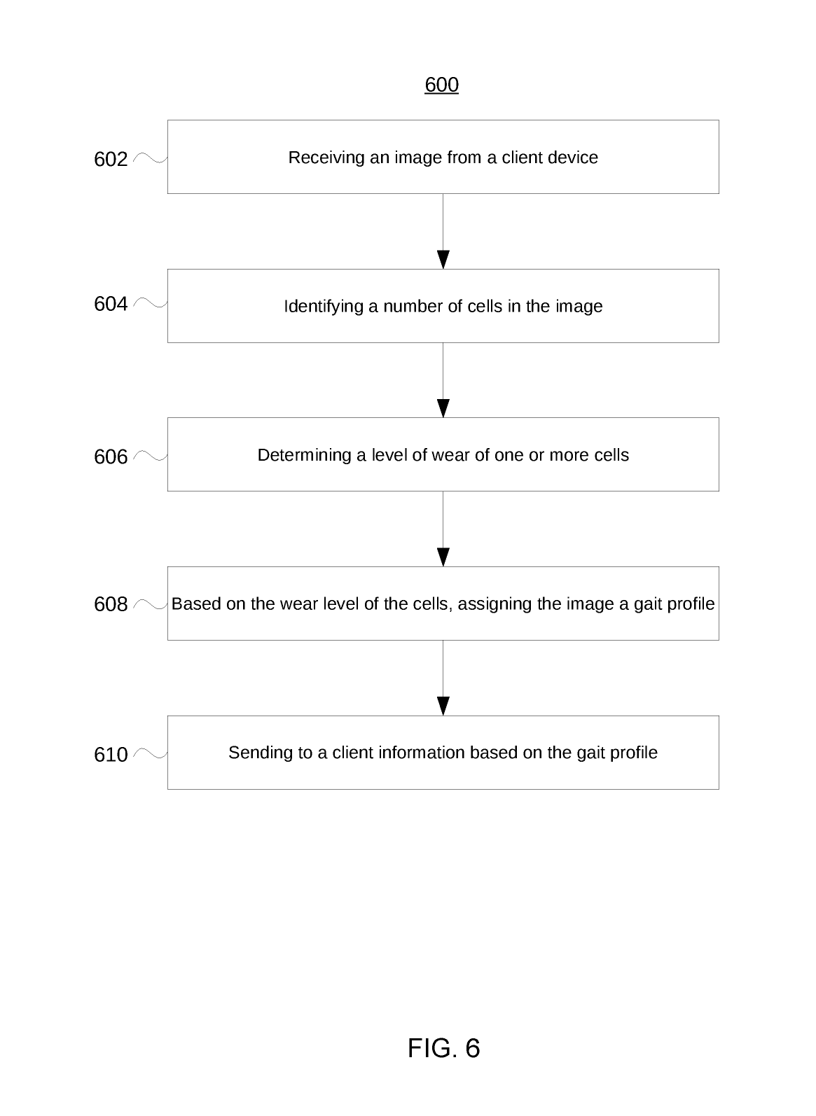

FIG. 6 shows an example method 600 for performing gait analysis using image processing of a shoe sole, according to an example embodiment. First, at step 602, gait analysis service 120 may receive an image of a shoe sole from a user, as described earlier. Gait analysis service 120 may then run an image processing method for determining the wear profile of the shoe. As an example, an image processing algorithm may start by identifying the cells in the shoe, as described in step 604. In an example, the image processing module scan the image to detect distinct squares of predetermined relative sizes or quantity, which are divided by ridges.

In particular embodiments, at step 606, gait analysis system 120 may further analyze the image to determine a level of wear. As an example, the image processing process may scan each cell to detect which cell segment borders are present and which are absent. As illustrated above with reference to FIG. 5, the image processing process may determine the level of wear of each cell based on which segment borders are visible and which are not. As an example, image processing process may determine whether a line in the image corresponding to a boundary between two segments is present or not, and if present, how thick or thin it is. Additionally, the image processing may determine the pattern of wear based on whether the line is partially or completely present. As an example, if the line is only partially present, the image processing process may infer that one portion of the line is wearing off more than another, and thus determine an angle of rotation of the foot.

Once the level and pattern of wear of each cell has been determined, at step 608, gait analysis service 120 may determine a gait profile associated with the image. A gait profile may be identified based on any combination of wear levels of any of the cells. As an example, gait analysis service 120 may determine that a runner has an overpronated gait based on a higher degree of wearing on a particular area of the sole. In an embodiment, gait analysis service 120 may represent the wear levels on the array of cells 310 as a vector, and map the vector to one or more vector spaces associated with a particular category or gait profile. In an embodiment, a gait profile may include any characteristic measurable or that can be inferred from the wear patterns of a sole, such as, by way of example, one or more gait types, conditions, running types, running frequency, or any combination thereof. In one example, characteristics to be included in a gait profile may be over-stride, heel strike, forefoot strike, or excessive rotation, normal stride, frequent runner, moderate runner, casual runner, shoes need replacement, abnormal pattern, etc. In particular embodiments, the profile may be determined based on a combination of wear patterns on both shoes, or may be determined separately for both shoes. While the determination of gait profile has been described in a particular manner, this disclosure contemplates the determination of a gait profile in any suitable manner.

At step 610, gait analysis 120 may cause delivery and/or the display of information associated with the determined gait pattern to a user. As an example, a user of client device 110 may receive gait analysis information, recommendations, data, etc. based on the submitted images. In particular embodiments, a user may referred to coaches that may help the user with any running/walking problems or concerns. In particular embodiments, gait analysis service 120 may deliver personally-tailored marketing materials, advertisements, offers, discounts, referrals, recommendations, etc. based on the gait analysis. For example, gait analysis system 120 may recommend particular new shoes for the user, or recommend particular ways of improving their gait.

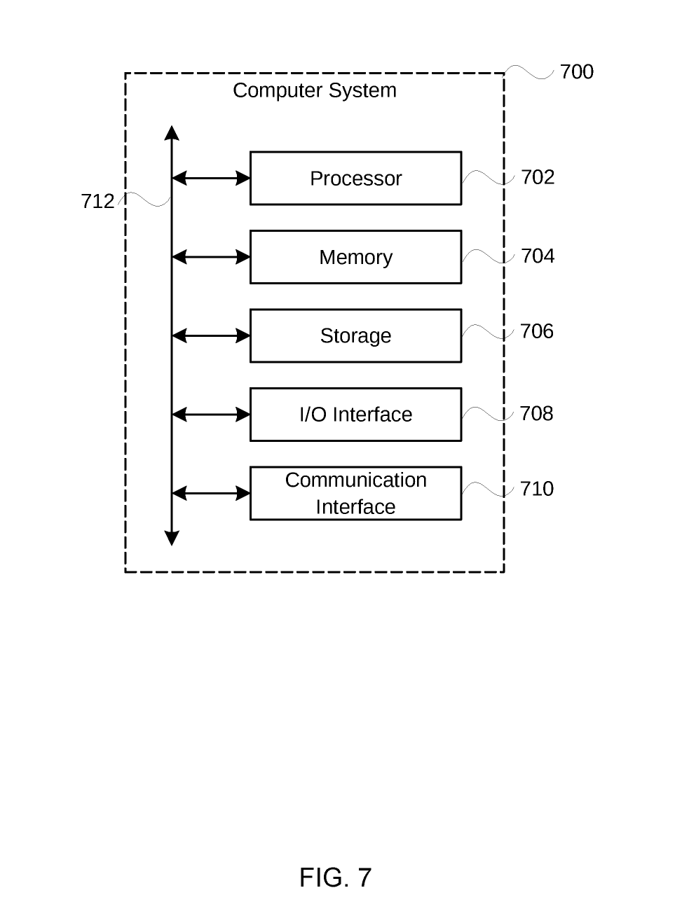

FIG. 7 illustrates an example computer system 700. In particular embodiments, one or more computer systems 700 perform one or more steps of one or more methods described or illustrated herein. In particular embodiments, one or more computer systems 400 provide functionality described or illustrated herein. In particular embodiments, software running on one or more computer systems 700 performs one or more steps of one or more methods described or illustrated herein or provides functionality described or illustrated herein. Particular embodiments include one or more portions of one or more computer systems 700. Herein, reference to a computer system may encompass a computing device, and vice versa, where appropriate. Moreover, reference to a computer system may encompass one or more computer systems, where appropriate.

This disclosure contemplates any suitable number of computer systems 700. This disclosure contemplates computer system 700 taking any suitable physical form. As example, computer system 700 may be an embedded computer system, a desktop computer system, a laptop or notebook computer system, a mainframe, a mobile telephone, a personal digital assistant (PDA), a server, a tablet computer system, or a combination of two or more of these. Where appropriate, computer system 700 may include one or more computer systems 700; be unitary or distributed; span multiple locations; span multiple machines; span multiple data centers; or reside in a cloud, which may include one or more cloud components in one or more networks. Where appropriate, one or more computer systems 700 may perform without substantial spatial or temporal limitation one or more steps of one or more methods described or illustrated herein. As an example, one or more computer systems 700 may perform in real time or in batch mode one or more steps of one or more methods described or illustrated herein. One or more computer systems 700 may perform at different times or at different locations one or more steps of one or more methods described or illustrated herein, where appropriate.

In particular embodiments, computer system 700 includes a processor 702, memory 707, storage 706, an input/output (I/O) interface 708, a communication interface 710, and a bus 712. Although this disclosure describes and illustrates a particular computer system having a particular number of particular components in a particular arrangement, this disclosure contemplates any suitable computer system having any suitable number of any suitable components in any suitable arrangement.

In particular embodiments, processor 702 includes hardware for executing instructions, such as those making up a computer program. As an example, to execute instructions, processor 702 may retrieve (or fetch) the instructions from an internal register, an internal cache, memory 704, or storage 706; decode and execute them; and then write one or more results to an internal register, an internal cache, memory 704, or storage 706. In particular embodiments, processor 702 may include one or more internal caches for data, instructions, or addresses. This disclosure contemplates processor 702 including any suitable number of any suitable internal caches, where appropriate. In particular embodiments, processor 702 may include one or more internal registers for data, instructions, or addresses. This disclosure contemplates processor 702 including any suitable number of any suitable internal registers, where appropriate. Where appropriate, processor 702 may include one or more arithmetic logic units (ALUs); be a multi-core processor; or include one or more processors 702. Although this disclosure describes and illustrates a particular processor, this disclosure contemplates any suitable processor.

In particular embodiments, memory 704 includes main memory for storing instructions for processor 702 to execute or data for processor 702 to operate on. As an example, computer system 700 may load instructions from storage 706 or another source (such as, for example, another computer system 700) to memory 704. Processor 702 may then load the instructions from memory 704 to an internal register or internal cache. To execute the instructions, processor 702 may retrieve the instructions from the internal register or internal cache and decode them. During or after execution of the instructions, processor 702 may write one or more results (which may be intermediate or final results) to the internal register or internal cache. Processor 702 may then write one or more of those results to memory 704. In particular embodiments, processor 702 executes only instructions in one or more internal registers or internal caches or in memory 704 (as opposed to storage 706 or elsewhere) and operates only on data in one or more internal registers or internal caches or in memory 704 (as opposed to storage 706 or elsewhere). One or more memory buses (which may each include an address bus and a data bus) may couple processor 702 to memory 704. Bus 712 may include one or more memory buses, as described below. In particular embodiments, memory 704 includes random access memory (RAM). This RAM may be volatile memory, where appropriate Memory 704 may include one or more memories 704, where appropriate. Although this disclosure describes and illustrates particular memory, this disclosure contemplates any suitable memory.

In particular embodiments, storage 706 includes mass storage for data or instructions. As an example, storage 706 may include a hard disk drive (HDD), a floppy disk drive, flash memory, an optical disc, a magneto-optical disc, magnetic tape, or a Universal Serial Bus (USB) drive or a combination of two or more of these. Storage 706 may include removable or non-removable (or fixed) media, where appropriate. Storage 706 may be internal or external to computer system 700, where appropriate. In particular embodiments, storage 706 is non-volatile, solid-state memory. In particular embodiments, storage 706 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 706 taking any suitable physical form. Storage 706 may include one or more storage control units facilitating communication between processor 702 and storage 706, where appropriate. Where appropriate, storage 706 may include one or more storages 706. Although this disclosure describes and illustrates particular storage, this disclosure contemplates any suitable storage.

In particular embodiments, I/O interface 708 includes hardware, software, or both, providing one or more interfaces for communication between computer system 700 and one or more I/O devices. Computer system 700 may include one or more of these I/O devices, where appropriate. One or more of these I/O devices may enable communication between a person and computer system 700. As an example, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 708 for them. Where appropriate, I/O interface 708 may include one or more device or software drivers enabling processor 702 to drive one or more of these I/O devices. I/O interface 708 may include one or more I/O interfaces 708, where appropriate. Although this disclosure describes and illustrates a particular I/O interface, this disclosure contemplates any suitable I/O interface.

In particular embodiments, communication interface 710 includes hardware, software, or both providing one or more interfaces for communication (such as, for example, packet-based communication) between computer system 700 and one or more other computer systems 700 or one or more networks. As an example, communication interface 710 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 710 for it. As an example, computer system 700 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 700 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 700 may include any suitable communication interface 710 for any of these networks, where appropriate. Communication interface 710 may include one or more communication interfaces 710, where appropriate. Although this disclosure describes and illustrates a particular communication interface, this disclosure contemplates any suitable communication interface.

In particular embodiments, bus 712 includes hardware, software, or both coupling components of computer system 700 to each other. As an example, bus 712 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 712 may include one or more buses 712, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

It is to be appreciated that the Detailed Description section, and not the Summary and Abstract sections (if any), is intended to be used to interpret the claims. The Summary and Abstract sections (if any) may set forth one or more but not all exemplary embodiments of the invention as contemplated by the inventor(s), and thus, are not intended to limit the invention or the appended claims in any way.

While the invention has been described herein with reference to exemplary embodiments for exemplary fields and applications, it should be understood that the invention is not limited thereto. Other embodiments and modifications thereto are possible, and are within the scope and spirit of the invention. For example, and without limiting the generality of this paragraph, embodiments are not limited to the software, hardware, firmware, and/or entities illustrated in the figures and/or described herein. Further, embodiments (whether or not explicitly described herein) have significant utility to fields and applications beyond the examples described herein.

Embodiments have been described herein with the aid of functional building blocks illustrating the implementation of specified functions and relationships thereof. The boundaries of these functional building blocks have been arbitrarily defined herein for the convenience of the description. Alternate boundaries can be defined as long as the specified functions and relationships (or equivalents thereof) are appropriately performed. Also, alternative embodiments may perform functional blocks, steps, operations, methods, etc. using orderings different than those described herein.

References herein to “one embodiment,” “an embodiment,” “an example embodiment,” or similar phrases, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of persons skilled in the relevant art(s) to incorporate such feature, structure, or characteristic into other embodiments whether or not explicitly mentioned or described herein.

The breadth and scope of the invention should not be limited by any of the above-described exemplary embodiments, but should be defined only in accordance with the following claims and their equivalents.

What is claimed is:

1. A computer implemented method comprising, by at least one processor:
receiving an image associated with a shoe sole, the image displaying one or more cells forming part of a tread pattern of the shoe sole;
determining, based on the image, one or more wear levels of at least one cell of the one or more cells;
analyzing the one or more wear levels to determine a wear pattern of the shoe sole; and
transmitting information associated with the wear pattern for display in an interface of a computing device.

2. The method of claim 1, wherein the determining one or more wear levels of at least one cell comprises:
determining a wear level of one or more segments of the at least one cell, wherein at least one of the segments has a different height than an adjacent segment.

3. The method of claim 2, wherein the determining a wear level of the one or more segments comprises determining the appearance of a line in the image corresponding to a boundary between the at least one of the segments and the adjacent segment.

4. The method of claim 2, wherein the one or more segments comprises:
a first segment with a first boundary in a first direction;
a second segment with a second boundary in a second direction, the second direction being orthogonal to the first direction; and
a third segment with a third boundary in a third direction, the third direction being diagonal to the first and second directions.

5. The method of claim 1, wherein the one or more cells forming part of a tread pattern of the shoe sole comprise a grid of equal-sized cells.

6. The method of claim 1, further comprising:
determining, based on the wear pattern, one or more gait categories associated with the shoe sole.

7. The method of claim 6, wherein the one or more gait categories comprises at least one of over-stride, heel strike, forefoot strike, or excessive rotation, or normal stride.

8. The method of claim 6, wherein the information associated with the wear pattern comprises the one or more gait categories or a marketing information associated with the one or more gait categories.

9. A system, comprising:
a memory; and
at least one processor coupled to the memory and configured to:
receive an image associated with a shoe sole, the image displaying one or more cells forming part of a tread pattern of the shoe sole;
determine, based on the image, one or more wear levels of at least one cell of the one or more cells;
analyze the one or more wear levels to determine a wear pattern of the shoe sole; and
transmit information associated with the wear pattern for display in an interface of a computing device.

10. The system of claim 9, wherein to determine one or more wear levels of at least one cell the at least one processor is further configured to:
determine a wear level of one or more segments of the at least one cell, wherein at least one of the segments has a different height than an adjacent segment.

11. The system of claim 10, wherein to determine a wear level of the one or more segments the at least one processor is further configured to determine the appearance of a line in the image corresponding to a boundary between the at least one of the segments and the adjacent segment.

12. The system of claim 10, wherein the one or more segments comprises:
a first segment with a first boundary in a first direction;
a second segment with a second boundary in a second direction, the second direction being orthogonal to the first direction; and
a third segment with a third boundary in a third direction, the third direction being diagonal to the first and second directions.

13. The system of claim 9, wherein the one or more cells forming part of a tread pattern of the shoe sole comprise a grid of equal-sized cells.

14. The system of claim 9, wherein the at least one processor is further configured to:
determine, based on the wear pattern, one or more gait categories associated with the shoe sole.

15. A tangible computer-readable device having instructions stored thereon that, when executed by at least one computing device, causes the at least one computing device to perform operations comprising:
receiving an image associated with a shoe sole, the image displaying one or more cells forming part of a tread pattern of the shoe sole;
determining, based on the image, one or more wear levels of at least one cell of the one or more cells;
analyzing the one or more wear levels to determine a wear pattern of the shoe sole; and
transmitting information associated with the wear pattern for display in an interface of a computing device.

16. The computer-readable device of claim 15, wherein the determining one or more wear levels of at least one cell comprises:
determining a wear level of one or more segments of the at least one cell, wherein at least one of the segments has a different height than an adjacent segment.

17. The computer-readable device of claim 16, wherein the determining a wear level of the one or more segments comprises determining the appearance of a line in the image corresponding to a boundary between the at least one of the segments and the adjacent segment.

18. The computer-readable device of claim 16, wherein the one or more segments comprises:
a first segment with a first boundary in a first direction;
a second segment with a second boundary in a second direction, the second direction being orthogonal to the first direction; and
a third segment with a third boundary in a third direction, the third direction being diagonal to the first and second directions.

19. The computer-readable device of claim 15, wherein the one or more cells forming part of a tread pattern of the shoe sole comprise a grid of equal-sized cells.

20. The computer-readable device of claim 15, the operations further comprising:
determining, based on the wear pattern, one or more gait categories associated with the shoe sole.
